(19) United States
(12) Patent Application Publication
(10) Pub. No.: US 2024/0108424 A1
(43) Pub. Date: Apr. 4, 2024

(54) ROBOTIC SYSTEM FOR DELIVERING ENERGY TO AN ANATOMICAL ELEMENT

(71) Applicant: CSA Medical, Inc., Lexington, MA (US)

(72) Inventors: Thomas Mulcahey, Belmont, MA (US); Wendelin Maners, Hermosa Beach, CA (US)

(21) Appl. No.: 18/480,274

(22) Filed: Oct. 3, 2023

Related U.S. Application Data
(60) Provisional application No. 63/412,606, filed on Oct. 3, 2022.

(51) Int. Cl. A61B 34/30; A61B 17/00; A61B 34/10; G06T 17/00; G16H 20/17

(52) U.S. Cl. CPC A61B 34/30; A61B 17/00; A61B 34/10; G06T 17/00; G16H 20/17; A61B 2017/00092; A61B 2034/107; A61B 2034/301; G06T 2207/10072

(57) ABSTRACT

Exemplary embodiments pertain to a therapeutic catheter for treating airway disorders. The catheter may include a video imager, steerable tip, and a lens temperature controller. The catheter may be remotely and/or robotically steerable based on a treatment plan developed with reference to a two- or three-dimensional map of the treatment area. Instead of individual discrete doses, regions of the treatment area may be treated in a continuous fashion by moving the catheter as a dose is applied. The catheter may be dynamically positioned at target locations in the lumen based on the treatment plan.

### CROSS-REFERENCE TO RELATED APPLICATIONS

This application claims priority to U.S. Provisional Patent Ser. No. 63/412,606, filed on Oct. 3, 2022 and entitled “Robotic System for Delivering Metered Energy to an Airway.” The contents of the aforementioned application are incorporated herein by reference in their entirety.

### BACKGROUND

The conducting airways of humans are lined by a superficial layer of epithelial cells which comprise an important primary line of defense to the entire respiratory tract. This superficial cellular layer consists primarily of mucus-producing (goblet) cells and ciliated cells. These cells function in a coordinated fashion to entrap inhaled biological and inert particulates and remove them from the airways. While this “mucociliary escalator” functions with great efficiency in the face of potentially injurious stimuli, it is a delicately balanced system relying on maintenance of appropriate complements of ciliated and mucus-producing cells and the normal functioning of those cells to accomplish effective clearance. Perturbations in epithelial cell type distribution and function can lead to adverse health effects.

Ciliated cells represent approximately 80% of the epithelial cells residing on luminal borders of the large airways. While they are the most prevalent epithelial cell type lining the airways, many studies suggest that they also are among the most vulnerable to injury by infection, irritant, and pollutant exposure. The identifying characteristic of ciliated cells, are the highly organized appendages of the cell, i.e., the cilia which cover the luminal border.

Mucus and other non-ciliated cells represent approximately 20% of the epithelial cells lining the luminal borders of the large airways. Mucus cells often are distended with secretory product and exhibit a characteristic “goblet” shape. Together with the submucosal glands, goblet cells secrete high molecular weight mucus glycoproteins (mucins). Goblet cells are thought to have the potential to produce markedly more mucus than do the glands, especially in response to injury such as environmental pollutants and other noxious elements such as tobacco/cigarette smoke.

Other non-ciliated cells with fewer or no granules also may be present along the luminal border. These may represent mucus cells which have emptied their contents onto the luminal surface or cells which have not yet differentiated. The entire epithelial layer sits on a basement lamina comprised of collagen and connective tissue. All the cells of the epithelial layer are anchored to this “basement membrane.”

Chronic bronchitis is a non-infectious inflammatory disease typically resulting from airway injury due to a noxious element (usually smoking). It is defined by cough with productive sputum of three months duration for two consecutive years. It is further characterized by excess mucus (mucus hyperactivity/hypersecretion/hyperplasia of goblet cells) in the bronchi, damage to cilia and loss of ciliated cells. Noxious stimuli lead to airway inflammation with swelling of the lamina propria leading to thickening of the airway wall, and this functional narrowing causes shortness of breath. More specifically, this injury causes over-proliferating goblet cells to over-produce a thick viscous, acidic mucus which is difficult to clear due to cilia dysfunction. The acidic mucous in chronic bronchitis leads to inflammation of the airway wall and varies in viscosity.

Asthma is a chronic respiratory disease characterized by bronchial inflammation, increased airway smooth muscle and airway hyper-responsiveness, in which airways narrow (constrict) excessively or too easily in response to a stimulus. Asthma episodes or attacks cause narrowing/constriction of the airways, which makes breathing difficult. Asthma attacks may occur at irregular intervals and be triggered by allergens or irritants that are inhaled into the lungs or by stress, cold air, viral infections or other stimuli. Asthma is sometimes, but not always, associated with mucus hyperactivity.

Airway hypersecretion is a feature of other airway diseases as well, including chronic obstructive pulmonary disease (COPD), cystic fibrosis, viral bronchitis, and bronchiolitis. COPD may be further broken down into two phenotypes: emphysema and chronic bronchitis. Symptoms of chronic bronchitis (CB) include increased sputum production (mucus hypersecretion) and/or persistent cough.

In an individual suffering from hypersecretion, mucus accumulates in the airways and may cause airway obstruction. Airway submucosal glands and goblet cells lining the airway epithelium secrete mucus, an adhesive, viscoelastic gel composed of water, carbohydrates, proteins, and lipids. In a healthy individual, mucus is a primary defense against inhaled foreign particles and infectious agents and is cleared by active columnated cilial cells/movement which assists in clearing the mucus in an upward direction where it is either swallowed or eliminated via a productive cough. Mucus traps these particles and agents and facilitates their clearance while also preventing tissues from drying out. Small airways that contain goblet cells as well as peripheral airways and which cannot be cleared by cough are particularly vulnerable to mucus accumulation and gradual obstruction by mucus.

Conventional treatments for individuals suffering from airway hypersecretion or chronic bronchitis include use of systemic or inhaled corticosteroids, anticholinergics, antibiotic therapy, bronchodilators (e.g., methylxanthines), short or long-acting beta2-agonists which relax the muscles in the airways to relieve symptoms, aerosol delivery of “mucolytic” agents (e.g., water, hypertonic saline solution), and oral administration of expectorants (e.g., guaifenesin). It should be noted that while these medications are variably approved by the FDA for use in COPD they are not specific for chronic bronchitis with the exceptions of roflumilast, an inhibitor of an enzyme called phosphodiesterase type 4 (PDE-4), and Trelegy Ellipta, an inhaler combining a LAMA, a LABA, and an inhaled corticosteroid (triple therapy).

Many of the above described medications have serious side effects. For example, inhaled corticosteroids can cause thrush (a yeast infection of the mouth), cough, or hoarseness, and systemic corticosteroids have even more severe side effects, such as delayed sexual development, changes in menstrual cycle, weight gain, and increased blood sugar (diabetes). The side effects of methylxanthines include severe nausea, tremors, muscle twitching, seizures, and irregular heartbeat. Roflumilast commonly induces significant diarrhea. Patient compliance is often low due to these side-effects.

Interventional approaches to managing occluded airways include surgery, mechanical debulking, brachytherapy, stents, bronchial rheoplasty, photodynamic therapy, and thermal modalities, such as electrocautery, laser, argon plasma coagulation, and bronchial thermoplasty. Bronchial thermoplasty is a procedure designed to help control severe asthma by reducing the mass of airway smooth muscle by delivering thermal energy to the airway wall, heating the tissue in a controlled manner. Bronchial thermoplasty with RF energy creates a deep ablation effect down to the level of the airway smooth muscle creating a reparative healing that results in scar tissue which is fibrotic in nature. Hyper-thermal treatment denatures proteins, and causes enyzme inactivation and prevents collagen remodeling. Accordingly, bronchial thermoplasty patients cannot be re-treated in the same areas. Cryoprobes have also been used in airway management, but their use can be tedious and time-consuming because of surface area limitations of the probes, which requires contact between the probe and the surface of the targeted lesion or tissue.

Positive outcomes have been reported from the use of Metered Cryospray (MCS) to selectively ablate and repopulate bronchial epithelium in patients with chronic bronchitis (Garner, et al. 2020), characterized by an improvement in quality of life scoring metrics such as SGRQ and CAT. Previously, Slebos, et al. (2017) reported evidence of bronchial resurfacing in human lobectomy patients post-MCS, confirming effective ablation penetrating into the submucosa and resulting in epithelial sloughing within 2 hours and complete epithelial regeneration within 14 days, leaving no evidence of fibrosis or impact to the underlying cartilage structures. The tissue at Day-14 was characterized as healthy, with functional cilia and normally distributed goblet cell populations. Orton, et al. (2020) further studied the transcriptomic response of chronic bronchitis patients to metered cryospray post-treatment and found that genes associated with epithelial remodeling exhibited elevated expression through at least 3-months following MCS. Based on published evidence, it has been concluded that metered cryospray is a safe and effective means of reducing cough and sputum production in COPD patients exhibiting the chronic bronchitis phenotype.

Emerging reports by Criner, et al. (2020) indicate that pulsed field ablation (PFA, non-thermal energy, pulsed electric field, PEF, bronchial rheoplasty, irreversible electroporation, reversible electroporation, dielectrophoresis) may be capable of creating a similar non-fibrotic healing response to MCS, and therefore may have similar effectiveness in treating central airway hyperplasia. PFA applies pulsed electrical energy packets via a contact electrode to the epithelium, creating a voltage potential which destabilizes the cell membrane (either temporarily or permanently), allowing extracellular electrolytes and water to freely transfer into the cell leading to organelle damage and apoptosis. PFA differs from similar modalities such as radiofrequency ablation (RFA) in that the voltage potential is pulsed at a frequency that allows heat to dissipate between wave packets, applying the voltage effect without raising the tissue temperature above the threshold for protein denaturation, which would in turn cause a fibrotic or reparative response as the extracellular matrix is damaged. As with MCS, PFA would indeed also benefit from the disclosed invention by applying energy with greater precision and speed than would be possible under physician-guided bronchoscopy, assuring more complete and continuous epithelial resurfacing.

Resector balloon desobstruction is an emerging technique (Karakoca, et al. 2015) that aims to abrade the central airway epithelium using a mesh-coated balloon which is repeatedly inflated/deflated, rotated, or translated in order to mechanically strip ciliated and goblet cells from the basement membrane, thereby also generating a repopulation response. While there is no effectiveness available as of this publication, the technique would most certainly benefit from the treatment planning and dynamic navigation methods disclosed herein.

### BRIEF SUMMARY

Exemplary embodiments pertain to a therapeutic catheter for treating airway disorders such as COPD. The catheter may include a video imager, steerable tip (e.g., via wire actuation and/or magnetic guidance), and a lens temperature controller (to avoid fogging to maintain clear visualization throughout a cryospray). In some embodiments, the catheter may further include an infrared imager for optimal dose spacing and thaw confirmation.

The therapeutic catheter may be robotically controlled. In some embodiments, a two- or three-dimensional representation of an airway may be obtained using (e.g.) CT scans and/or MRI imaging prior to therapy in order to facilitate treatment planning. The 3D scans may be used to create a detailed virtual map of the treatment area, which may include the trachea and first four segments of the bronchial tree (out to the segmental bronchi). The representation may allow cross-sectional analysis (transverse sectioning) of the lumens throughout the area of interest.

In order to optimize dose delivery and expedite procedure time, treatment planning software may identify all suitable segments that can be treated with metered cryospray (MCS) and may parse out the eligible segments and characterize them according to lumen diameter, circularity, and wall thickness. These parameters may be used by the planning software to determine optimal dose amounts at locations along each eligible segment.

In an automated fashion, the treatment planning software determines the optimal effective MCS dose for each cross section and creates a treatment plan which is then downloaded to an MCS console to execute the procedure. An MCS catheter may then be loaded into the navigation platform's steerable working channel, and the patient may be intubated. The robotic system, in communication with the MCS console, proceeds to the first treatment location and alerts the user of its readiness to treat. The MCS console, preloaded with the treatment parameters from the planning software, then executes a controlled energy delivery in the first location

During treatment, the robotic catheter may work with the MCS console to maintain centering according to the treatment plan throughout the spray application. The catheter may adjust to movement of the patient (through respiration, cardiac rhythm, etc.) to assure intended placement. The robotic catheter may be autonomously steered to center the spray nozzle based on visual and transducer feedback provided to a processor at the MCS console. The spray nozzle of the catheter may be autonomously centered based on visual and transducer feedback to the processor (e.g., through the catheter's video imager). The catheter may be automatically adjusted based on visual frost cues. The catheter may include multiple (e.g., 3) sensors for near-field distance measurements, which may be used to triangulate the spray nozzle's location in the body lumen and center it. Furthermore, sensor data from one or more transducers may be used to calibrate image processing. All this may be done without the need for visualization by a user and manual feedback.

The MCS console may include a user interface that shows an augmented reality (AR) overlay. The AR overlay may depict virtual fiducials of previous treatment locations as well as cues for the targeting center and axial placement of the catheter.

In order to reduce procedure time and improve ablation coverage such that more complete eradication of goblet cells and cilia is achieved, the robotic system may deliver doses in a traversing fashion (or pullback) rather than spacing discrete sprays. By continuously displacing the catheter during a spray, entire segments can be uniformly dosed, and traverse speed can be varied throughout the maneuver to account for variation in lumen diameter across the segment. To support this continuous pullback technique, a heater circuit may be added to the catheter's flexible circuit to allow isothermal energy measurement (similar to a hot wire anemometer).

This process also reduces procedure time, as the current MCS protocol may require a wait period (e.g., 30 seconds) between sprays to allow the system to reacclimate and the catheter's feedback thermocouple to return to a temperature above 20° C., such that it can meter energy using the temperature change data of the catheter during a spray. By modifying the catheter's feedback sensor to include a heating element, or alternatively by running electrical current through the thermocouple circuit to generate a heating effect in the Constantan leg of a T-type thermocouple, the sensor can be maintained at a steady temperature while still functioning as an energy meter using the first law of thermodynamics for a steady system, where the electrical energy input to the sensor is equal to the cooling energy being removed by the cryogen stream, thus allowing continuous use without thawing between applications.

Once the first site has been dosed, the navigation system then maneuvers to the next target and alerts the user that it is ready to apply the next dose. The user may initiate the next dose (or it may be initiated automatically) and the combined MCS/robotic system automatically delivers the next dose according to the treatment plan. This process repeats until all target segments have been dosed. By using this highly automated process, along with continuous pullback, each procedure is reduced by up to 15 minutes, decreasing the amount of time the patient is under anesthesia and making more efficient use of surgical facilities.

In addition to reducing procedure time, patient safety and MCS efficacy may be improved by assuring the dose is delivered in the geometric center of the lumen, reducing the risk that the catheter is biased to one side of the anatomy, which in turn reduces the risk of deeper penetration into structural tissues such as cartilage or adjacent organs or vasculature. As visualization may be challenging for MCS applied through a conventional bronchoscope due to frost or spatter accumulation on the lens during the spray application, the navigation technology assures centering is maintained even if visualization is lost. Safety is also improved due to the smaller outside diameter (OD) of navigated and robotic working channels compared to those of conventional bronchoscopes with similar working channel sizes, allowing improved gas egress and reducing the risk of barotrauma such as pneumothorax or pneumomediastinum. This smaller profile may also allow MCS to be delivered to smaller segments (e.g. <6-mm lumen diameter) which would otherwise be contraindicated due to flow restriction.

In one application example, chronic bronchitis (a phenotype of COPD) may be treated by ablating the trachea and bronchial mucosa in the first 3-4 generations using modalities that stimulate a rejuvenated response with minimal scarring, such as spray cryotherapy, contact cryotherapy, non-thermal ablation (pulsed electrical field (PEF), irreversible electroporation (IRE), dielectrophoresis), or any other modality that results in cellular death without stimulating a substantial fibrotic or reparative response.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a perspective view of a cryosurgery system according to an embodiment of the invention;

FIG. 2 is a perspective view of another embodiment of a cryosurgery system according to the invention;

FIG. 3 is a perspective view of the interior of an embodiment of a cryosurgery system according to an embodiment of the invention;

FIG. 4 is a schematic showing a cryogen storage, delivery and pressure control apparatus according to another embodiment of the invention;

FIG. 5A depicts an example of centering when cryospray is applied to a regular body lumen in a single pass, showing the amount of the lumen's injury zone that can be effectively treated.

FIG. 5B illustrates an example of centering a two-pass cryospray treatment.

FIG. 5C illustrates an example of a three-pass cryospray treatment.

FIG. 5D illustrates an example of a five-pass cryospray treatment.

FIG. 6A depicts an example of a single-pass cryospray treatment in an irregularly-shaped body lumen.

FIG. 6B illustrates an example of a multiple-pass cryospray treatment in an irregularly-shaped body lumen.

FIG. 6C is a flowchart depicting an exemplary method for delivering discrete doses of cryogenic fluid to a patient.

FIG. 7A depicts an example of a continuous transverse spray.

FIG. 7B depicts an example of a velocity applied to the catheter tip over time as part of a continuous transverse spray as determined by a treatment plan.

FIG. 8A illustrates an example of catheter centering when applied to a body lumen with a hazard in the form of eccentric structural cartilage.

FIG. 8B illustrates an aspect of the subject matter in accordance with one embodiment.

FIG. 9 illustrates an example of a longitudinal traverse of a robotic catheter according to a treatment plan.

FIG. 10 depicts an example of a catheter in accordance with one embodiment.

FIG. 11 depicts an optional heater for the catheter of FIG. 10 in accordance with one embodiment.

FIG. 12 depicts another embodiment of an optional heater for the catheter of FIG. 10.

FIG. 13 depicts an example of an optimized traversal path for the catheter in a branch of varying radii.

FIG. 14 depicts a catheter traversing the optimized traversal path of FIG. 13 and a plot of path velocity for the catheter over time.

FIG. 15 depicts the end components of an illustrative catheter of an exemplary embodiment.

FIG. 16 depicts examples of augmented reality overlays that may be provided in an exemplary embodiment to aid in catheter positioning.

### DETAILED DESCRIPTION

Exemplary embodiments pertain to a therapeutic catheter for treating airway disorders. The catheter may include a video imager, steerable tip, and a lens temperature controller. The catheter may be remotely and/or robotically steerable based on a treatment plan developed with reference to a two- or three-dimensional map of the treatment area. Instead of individual discrete doses, regions of the treatment area may be treated in a continuous fashion by moving the catheter as a dose is applied. The catheter may be dynamically positioned at target locations in the lumen based on the treatment plan.

The treatment planning software may use a two- or three-dimensional map to divide the area to be treated into regions that may be treated. For instance, the treatment planning software may identify certain structures, such as branch points, which must be avoided due to the risk of damaging them (e.g., due to the tissue in this area being thin or the cartilage being irregularly-shaped). Furthermore, the patient's oxygen level must be managed, which means limiting ventilator detachment to a reasonable duration (e.g. 90 sec) and/or responding to SpO2 (blood oxygen saturation) levels. The treatment planning software may account for these constraints when breaking up the overall treatment area into smaller treatment regions. The software may be configured to select some or all of these treatment areas for continuous dosing.

The treatment planning software may determine a calculated dose amount of ablative energy for each treatment region. As noted above, the treatment planning software avoids bifurcations/trifurcations between branch generations and other critical structures. Furthermore, in areas that are treated, each dose of ablative energy should be limited to a predetermined length of time to manage the patient's oxygen level (e.g. 90 seconds). Still further, the system may attempt to find an optimal treatment plan which minimizes overall procedure time and risk to the underlying structure, while maximizing surface area coverage and freeze depth.

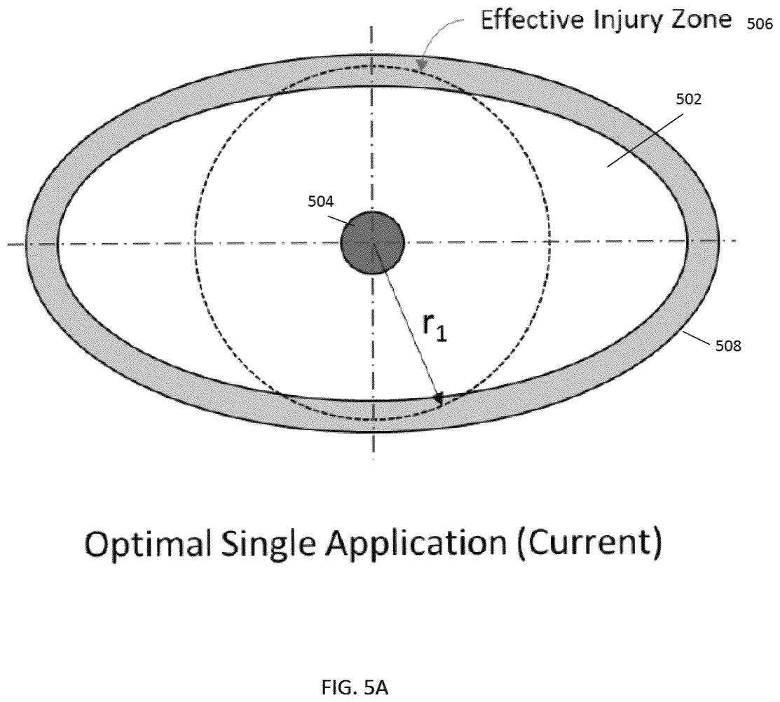

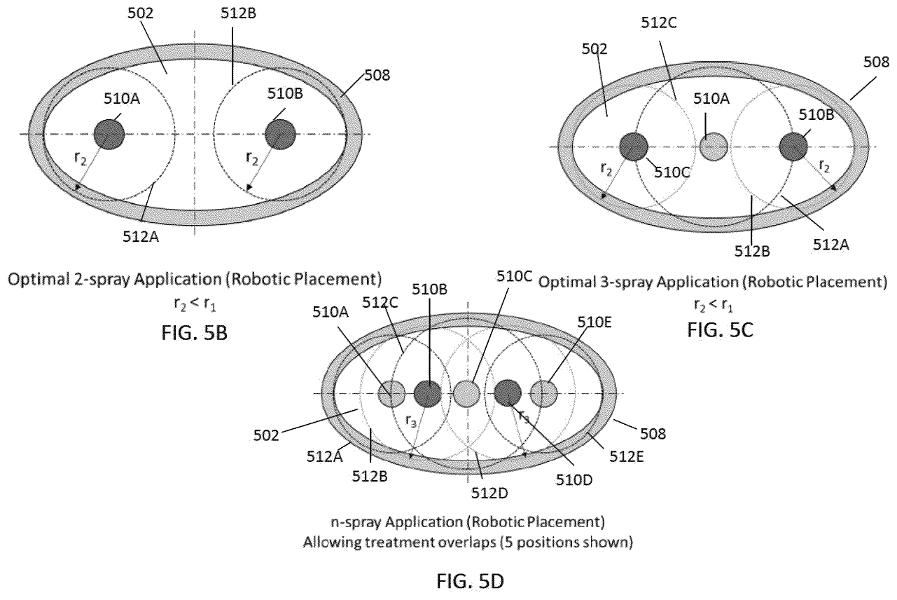

Within these constraints, the planning software may attempt to determine an optimal center for each transverse cross section along the path which maximizes perimeter coverage, while avoiding buried structures. Note that the “center” location in this context refers to the center of the catheter, not necessarily requiring that the catheter be centered in the body lumen. In fact, the catheter “centering” may be optimized to a location which is not the geometric center of the body lumen in order to maximize uniformity. An example of this would be an oval-shaped cross-section, which may be optimized between the center and the foci, and treated in two separate applications in order to maximize the surface coverage (see, e.g., FIG. 5B). Compared to a single application in the geometric center, applying two discrete sprays would increase perimeter coverage (with the improvement increasing for further discrete sprays).

The planning software may determine a dosing strategy based on applying discrete sprays, and/or may apply ablative energy, such as in the form of a cryospray, continuously over the region to be treated. It should be appreciated the exemplary embodiments are not limited to cryospray application; rather other forms of ablative energy may be applied. Continuous applications may be applied along the axis of the lumen (e.g., in the above-noted “pullback” method), and/or along a longitudinal traverse path. An optimal longitudinal traverse path may be found by 3-D interpolation between each optimized transverse center (e.g., as a smooth arc).

Benefits of the exemplary embodiments may include, but are not limited to:
Precise placement and dosimetry
Robotic workflow collects high resolution CT scans (HRCT) along with planning software to define the navigation path through a 3D virtual model
Planning software can assess cross-sectional lumen profiles at each target location along the airway branch, selecting the optimal effective diameter
   Customizes treatment for each patient rather than using published guidelines for male/female anatomy
   Accounts for non-circular profile of airways
   Accounts for presence of structural anatomy (e.g. cartilage near carinas, particularly thin epithelial areas)
Ideal centering
   Robotic workflow collects high resolution CT scans (HRCT) along with planning software to define the navigation path through a 3D virtual model
   Planning software can assess cross-sectional lumen profiles at each target location along the airway branch, selecting the optimal centering location to account for non-circularity
   Robotic controls automatically maintain center as the patient moves (respiration/cardiac rhythm)
Improved gas egress
   Navigated working channel has typical OD of 3-4 mm, compared to pediatric/diagnostic bronchoscopes with OD of 4.2 to 5 mm
   Increased area allows smaller endotracheal tube (for difficult intubations)
   Decreased OD allows treatment of smaller segments (currently limited to 6 mm or greater)

These and other features will now be described with reference to the attached figures. Note that, although the discussion below focuses on a cryospray apparatus, the invention is not limited to this specific delivery methodology. Spraying a cryogenic fluid (e.g., a fluid having a flow boiling point of, e.g., −130 F or below, such as liquid nitrogen) is one technique for delivering energy to tissue. Other types of energy, such as different types of thermal energy, radio frequency energy, laser energy, non-thermal ablation energy, a pulsed electrical field, irreversible electroporation, dielectrophoresis, mechanical energy, etc. may also be applied as appropriate.

### Apparatus

For illustration purposes, an exemplary cryotherapy apparatus is first described. It is noted, however, that the present disclosure is not limited to the application of cryogen, but more generally applies to any suitable type of energy transfer.

Certain methods and devices described herein are improvements to the methods and devices described U.S. patent application Ser. No. 13/784,596, filed Mar. 4, 2013, entitled “Cryosurgery System,” U.S. patent application Ser. No. 14/012,320, filed Aug. 28, 2013, and U.S. patent application Ser. No. 14/731,359, filed Jun. 4, 2015, entitled “Method and system for consistent, repeatable, and safe cryospray treatment of airway tissue.”

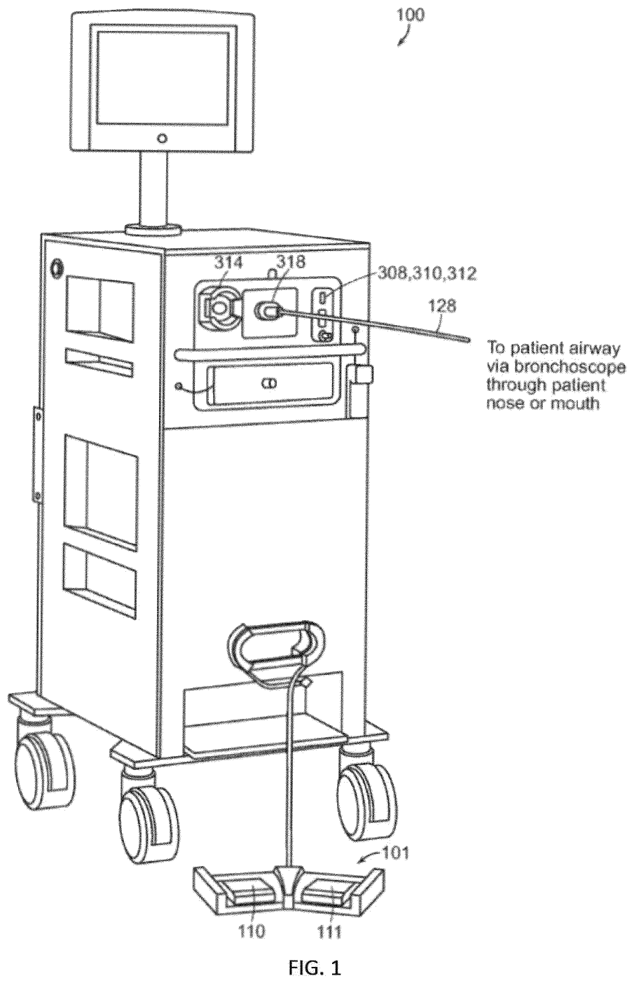

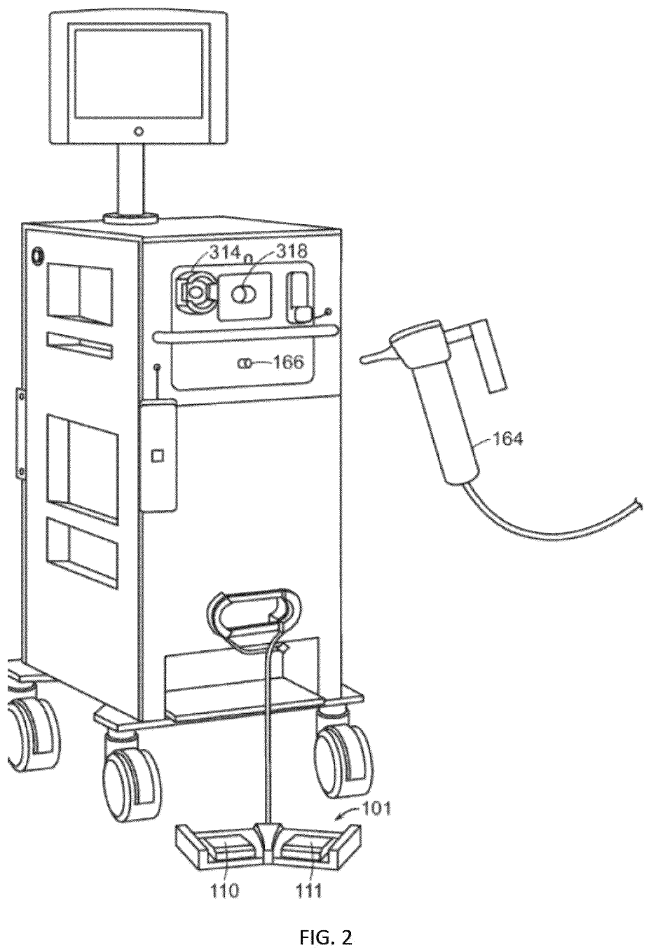

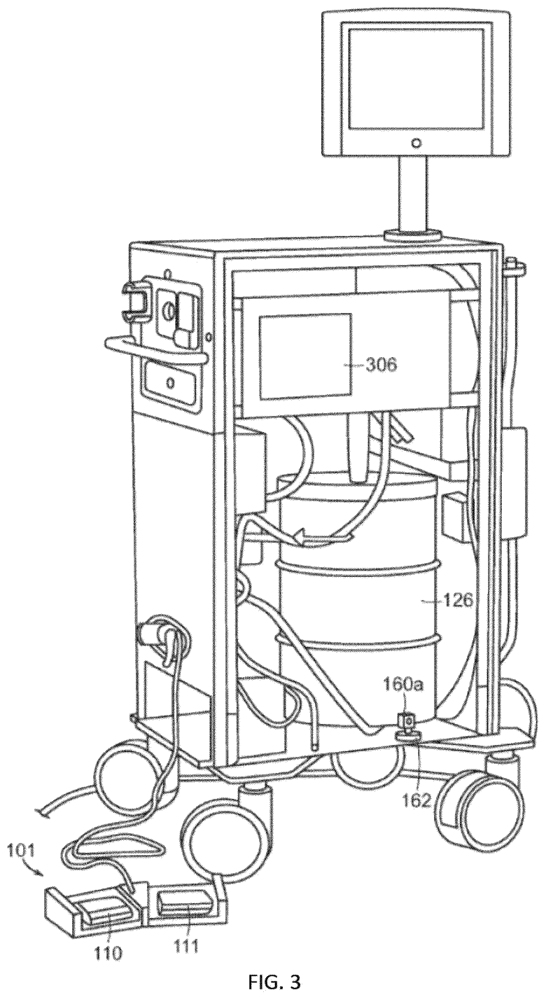

A simplified perspective view of an exemplary cryosurgery system in which embodiments of the present invention may be implemented is illustrated in FIGS. 1-3. Cryosurgery system 100 comprises a pressurized cryogen storage tank 126 to store cryogen under pressure. In the following description, the cryogen stored in tank 126 is liquid nitrogen although cryogen may be other materials as described in detail below. The pressure for the liquefied gas in the tank may range from 5 psi to 50 psi. According to a more preferred embodiment, pressure in the tank during storage is 40 psi or less, and pressure in the tank during operation is 35 psi or less. According to a more preferred embodiment, pressure in the tank during storage is 35 psi or less and pressure during operation is 25 psi or less. According to a most preferred embodiment, pressure during operation at normal nitrogen flow is 20±4 psi.

Nominal tank pressures according to preferred embodiments of the present invention are established to assure that different systems have a standardized energy output, which is, for example, the nominal energy output of a standard system used to successfully deliver treatment in an animal model or in a human patient according to one of the various embodiments of the present invention; Energy output of individual systems is assessed using one or more of a standard catheter and/or a standard airway phantom comprising multiple one or more temperature sensing elements (e.g. one or more thermocouples); temperature changes measured by the phantom are used to calculate the total energy output during the spray, and multiple sprays may be carried out at varying pressures to establish a pressure-energy relationship that is then used to select a pressure value that yields the energy output of the standard system, within a predetermined error (e.g. ±5% of standard energy output).

In an alternate embodiment, the cryogen pressure may be controlled all the way to 45 psi to deliver through smaller lumen catheters and additional feature sets. In such alternate embodiments the pressure in the tank during storage may be 55 psi or less.

Liquid nitrogen (LN2) resides on the bottom of the tank and liquid nitrogen gas/vapor (GN2) occupies the top portion of the tank. Tank level is monitored electronically via a sensor internal to the tank that changes value with the level of the liquid inside the tank. This can be done in a variety of ways, including but not limited to capacitively (an example being a Rotarex C-Stic), resistively, or by measuring differential pressure.

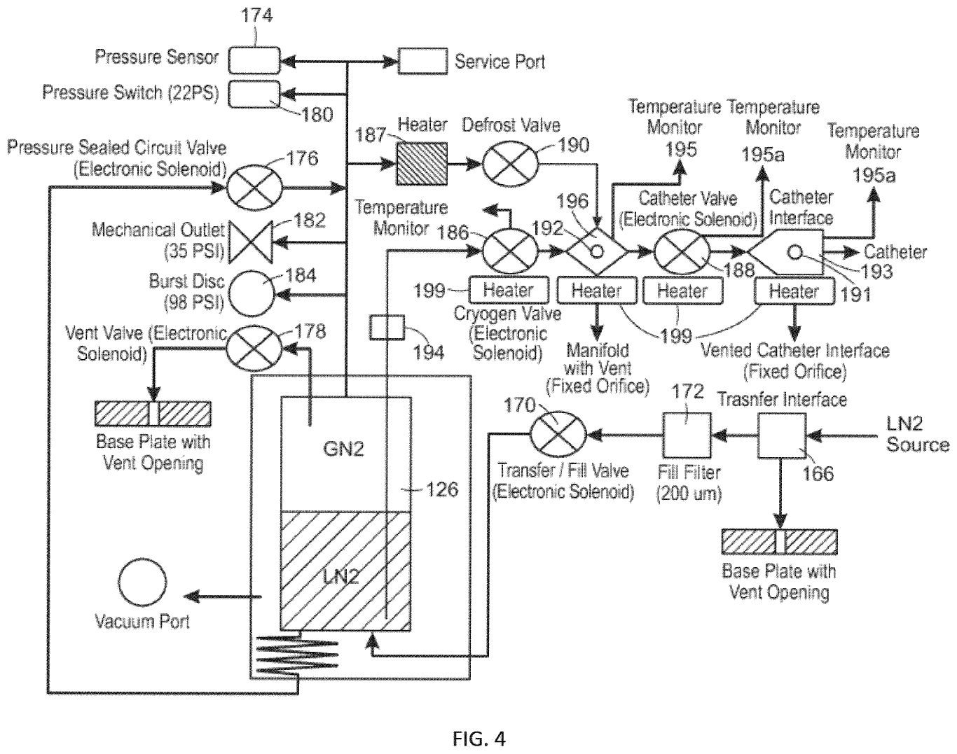

Referring to FIG. 4, the present invention utilizes valves and a pressure sensor 174 to continuously monitor and control the pressure of liquid nitrogen in the tank during use. The console monitors the current pressure of the tank via a pressure sensor 174. The software reads the current pressure from the sensor and adjusts the pressure accordingly. If pressure is too low, the software actuates the pressure build circuit valve 176 to increase the pressure to a specified threshold and then turns off. When the pressure is too high, the software turns on the vent valve 178 until the pressure reaches a specified threshold.

In some cases, system charge pressure is actively controlled by a set of three solenoid valves. A cryogenic solenoid valve connected to the head space is used for rough reduction of tank pressure in cases where tank pressure is significantly above the desired set pressure (>5 psi) or during fill operations when tank pressure must be completely relieved. A set of proportional solenoid valves control the pressure vent and pressure build functions. The proportional solenoid valves are driven by a pulse width modulation (PWM) controller which adjusts its duty cycle based on a control voltage, allowing the valve plunger position to open proportional to the control signal. The control signal is driven by a standard proportional integral derivative (PID) control algorithm executable by a central processor of the system. The PID controller collects data from a precision capacitive pressure sensor and adjusts the valve control signal based on the current pressure deviation with respect to the set point, the current rate of change of pressure, and the pressure history. A PID output control signal determines whether venting or building operations occur. This control scheme advantageously implements precise pressure regulation while allowing software changes to the pressure set point. The PID controller is tuned (inputs P, I, and D) to provide quick response with minimal overshoot or undershoot, while avoiding unstable cycling between vent and build operations.

A mechanical relief valve 182 on the console tank ensures that the tank pressure stays in a safe pressure range. Constant pressure monitoring and adjustment, allows the set point on the mechanical relief valve to be set at 35 psi, allowing for a low tank storage pressure. A redundant burst disk 184 provides protection should the mechanical relief valve fail. For optimal safety, both electronic and mechanical pressure valves are present to regulate the pressure, providing triple redundancy in the event of failure. In addition, a redundant pressure switch 180 may provide accurate tank pressure readings and is checked during the self-test. In an alternate embodiment, the mechanical relief valve 182 may be set at 60 psi, but still allowing to remain a low pressure storage tank.

The system of the present invention utilizes a manifold assembly including cryogen valve 186, manifold 196, catheter valve 188, defrost valve 190, fixed orifices 191 and 192, and catheter interface 193 to control liquid nitrogen delivered through the catheter. When the cryogen valve 186 is actuated, liquid nitrogen exits the tank through the lance 194 and proceeds through the cryogen valve 186 to manifold 196 where fixed orifice 192 is present to allow cold expanded gas and liquid cryogen to exit the line and cool down the internal cryogen circuit. During this precool, the catheter valve 188 downstream of the manifold remains closed. A data acquisition board collects data from a thermocouple 195 located on the manifold body. In the precool function, the system software monitors data from the thermocouple 195, and opens the cryogen valve 186 to cool the manifold 196 when its temperature is above the desired set-point. According to a preferred embodiment, fixed orifice 191 is provided on catheter interface 193 to allow venting of cold expanded gas to exit the line while spraying.

According to a preferred embodiment of the invention, represented in FIGS. 4, each of cryogen valve 186, manifold 192, catheter valve 188 and catheter interface 193 are provided with a temperature thermocouple or sensor 195 a and a heater 199 to maintain the cryogen flow path at a constant selected temperature to prevent overcooling of the system resulting from the continuous flow of cryogen through the valves and manifold assembly. According to various embodiments of the invention, each of the heaters may be controlled to maintain the valves, the manifold and the catheter interface at the same temperature or at different temperatures. According to a preferred embodiment, the system is set so that the temperature(s) of the valves, manifold, and catheter interface is/are controlled to be maintained at a temperature greater than −120° C. during cryospray treatment. According to a most preferred embodiment, the system is set so that the temperature(s) of the valves, manifold, and catheter interface is/are controlled to be maintained at a temperature of +20° C. during cryospray treatment. According to another embodiment, each of the valves, manifold, and catheter interface are controlled and maintained at constant temperatures, but the constant temperatures of each may be different from one or more of the constant temperatures of the others.

A defrost function is useful for thawing the catheter after cryogen spray, before removal from the scope. A defrost circuit directs gaseous nitrogen from the top of the tank through a heater 187 and defrost valve 190 to the catheter 128. When the defrost button on the software screen is pressed, the defrost circuit is activated for a prescribed time (e.g. 30 seconds) but can be stopped earlier at the user's discretion. A low voltage (24 VDC) DC defrost heater delivers 6 W minimum of warming/defrost performance but minimizes variation due to line voltage and limits maximum gas temperature, as compared to the prior art line voltage (120V) AC heater.

The console of the present invention comes with an insulated quick release custom fill hose 164 to fill the tank through the external fill port 166 in a semi-automatic cryogen fill process. A fill port switch on the console actuates only when the fill hose is in the locked position. During the fill process, liquid nitrogen passes through a filter 172 and transfer valve 170 en route to the tank. The software automatically shuts off the electronic transfer valve 170 when the tank is full and vents the hose prior to removing from the console. According to an alternate embodiment, manual filling can take place by mechanically bypassing the electronic transfer and vent valves with manual valves, thus allowing the tank to be filled without the need for computer control.

The catheter is designed to transport liquid nitrogen (or other cryogen) from the console to the patient treatment site. According to one embodiment, the catheter 1 may contain a bayonet 2 and hub 3 for attachment to the console at its proximal end, a laser cut hypotube to minimize kinking and breaking, and a polymer layer disposed over the hypotube, thereby sealing the catheter 1, and an insulation layer 4 to protect the user from cold, a strain relief 4 to help prevent kinking when torqued by users and an atraumatic rounded tip (10) at its distal end to prevent damage to tissue. The hypotube is preferably perforated using an interrupted spiral cut, imparting radial flexibility while maintaining some axial stiffness and pushability, and the relative flexibility of the hypotube is, in some cases, variable along the length of the catheter 1 through the use of a variable-pitch spiral cut. For instance, the spiral cut may be characterized by a first, relatively large pitch proximally, and a second, smaller pitch more distally, allowing the distal end, and particularly the tip, to bend about a tighter curve than the most proximal portions of the catheter. The strength and flexibility provided by catheters according to these embodiments allows a user (e.g. a physician or navigation system) to retroflex the catheter during a treatment procedure, if needed.

The polymer layer may be any suitable flexible polymer that is substantially gas impermeable (for example fluorinated ethylene propylene, urethane, or polyethylene terephthalate), and may be disposed over the hypotube in the form of one or more extrusion layers attached by means of heat shrinking, or by means of dip coating, melt coating or spray coating. The catheter package may contain an RFID tag or embedded authentication EEPROM that the user scans prior to use to prevent reuse and track disposable information.

The catheter package may also contain an introducer that provides reinforcement for the catheter and helps prevent kinking during use and when placing the catheter into the scope. An alternative construction locates the RFID tag or authentication EEPROM on the connector area adjacent to the bayonet, such that the authentication information is read by the system when the catheter is connected to the system.

According to a preferred embodiment, the delivery catheter may be constructed out of hypotubes of different internal diameters mated to each other to make a proximal shaft and a distal shaft, with the distal shaft containing the smaller ID. The proximal and distal shafts may be joined at a connector, which connector can be covered by a molded handle to permit a user to make fine adjustments to the catheter 1. The proximal shaft may contain a bayonet and hub for attachment to the console at its proximal end. The distal shaft preferably has a reduced ID to be able to fit through the working channels of a bronchoscope or steerable robotic catheter. The distal tip of the catheter contains the radial spray pattern holes which make up the nozzles configured to deliver the cryogen spray onto the target tissue. The end of the catheter may be configured to have rounded tip, preferably made of a welded stainless steel sphere. This rounded tip may help reduce trauma to the tissue during catheter insertion or manipulation into the body cavities. A thermocouple may be located along the catheter shaft, preferably at or near the distal tip of the catheter, to provide temperature feedback to the control console, for example to better determine the precise moment that cryospray exits the tip of the catheter and/or to monitor the net delivery of ablation energy throughout the dose application. The hypotubes are all laminated with a polymeric heatshrink which seals the laser cut pattern from the liquid intended to flow inside the catheter. Additionally, both hypotubes have variable laser cut patterns which provide rigidity where needed and much flexibility where needed. This is accomplished by varying the separation of the spiral or repeated cut pattern, as well as varying the shape of the pattern itself.

### Treatment Plans for Application by a Robotic Catheter

FIGS. 5A-5D depict examples of dosing strategies that may be applied by treatment planning software. For example, FIG. 5A depicts an example of a conventional dosing scheme in which the catheter 504 is maintained as close as possible to the geometric center of the lumen 502 of an airway 508. Typically, the centering is performed based on a generalized average lumen size calculation for the patient's gender and the airway segment being treated, which may only be accurate to ±30%. As can be seen, the result can be a relatively small amount of treatment area being covered in the case of an irregularly shaped cross section (see effective injury zone 506).

FIGS. 5B-5D show alternative treatment plans in which discrete doses are applied at two (FIG. 5B), three (FIG. 5C), or more (FIG. 5D) center locations. As shown in FIG. 5B, there are two spray locations 510A and 510B arranged in the lumen 502 of the airway. Each spray location, has a respective effective injury zone 512A, 512B. FIG. 5C depicts an example in which three spray locations 510A, 510B, and 510C are arranged in the lumen 502 of an airway 508 with effective injury zones 512A, 512B, and 512C. FIG. 5D depicts an example in which five pray locations 510A, 510B, 510C, 510D, and 510E are positioned in a lumen 502 of an airway. The respective injury zones 512A, 512B, 512C, 512D, and 512E are shown. In all of the arrangements shown in FIGS. 5B-5D, the spray locations may be located on a central horizontal axis at uniformly spaced locations to provide fuller spray coverage. When coupled with high resolution CT scans or MRI scans, the ability to robotically position a catheter precisely to apply multiple doses of energy within a given treatment area can mean significantly greater coverage.

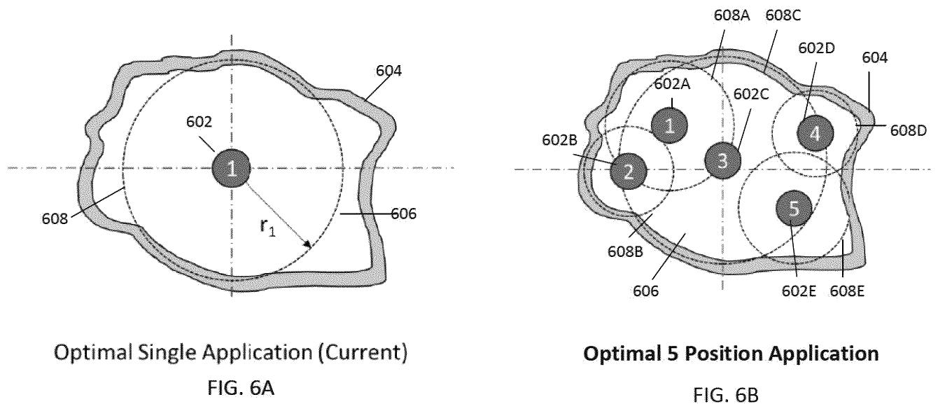

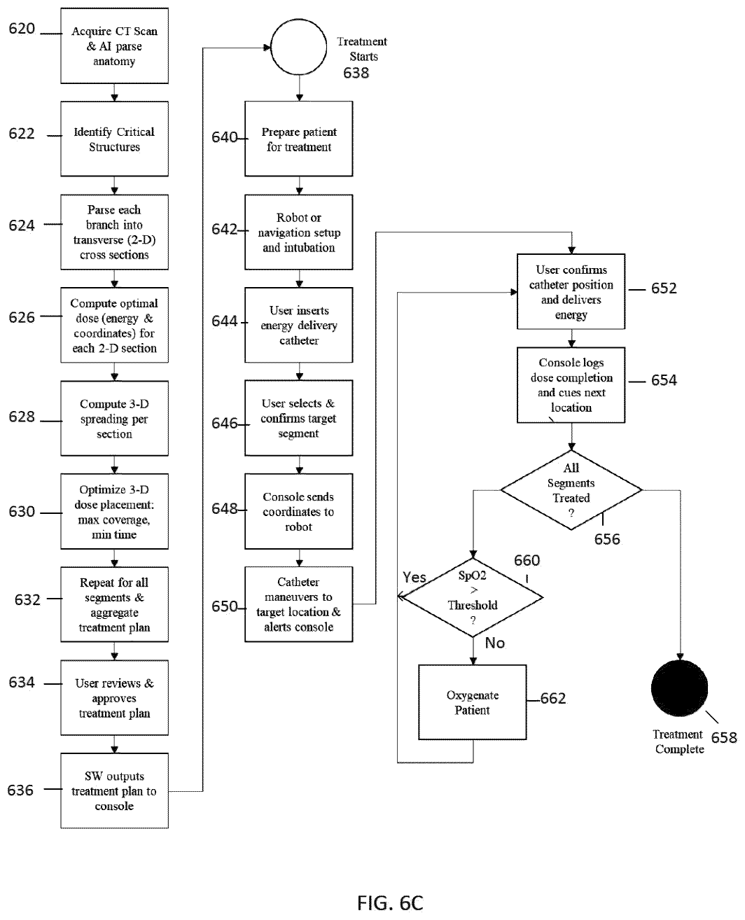

Performing multiple doses in different centered locations also means that more complicated body lumens can be treated more effectively and efficiently, as shown for example in FIGS. 6A-6B. In FIG. 6A a complicated lumen 606 of a body part 604. The spray location 602 is centrally located, resulting in effective injury zone 608. As can be seen, with the complicated geometry of the lumen 606, much of the portion of the body part is not reached by the spray. In contrast, in FIG. 6B, the use of the 5 spray locations 602A, 602B, 602C, 602D, and 602E in the lumen 606, covers more of the body part 604 surface as indicated by respective effective injury zones of 608A, 608B, 608C, 608D, and 608E.

FIG. 6C illustrates an example routine for delivering discrete doses of a cryogenic fluid to a patient, as might be applied in the examples shown in FIGS. 5A-6B. Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

Initially, at 620, a computed tomography (CT) scan of the relevant anatomy of the patient may be obtained, and artificial intelligence (AI) techniques may be applied to parse the anatomical features that are imaged in the CT scan. Critical structures in the anatomy may be identified at 622. Each branch avoiding critical structures may be parsed into transverse (2-D) cross-sections at 624. At 626, an optimal dose or near-optimal dose may be computed for each 2-D section. The dose may specify how much cryospray or ablation energy in alternative embodiments is to be applied to the 2-D section. At 628, a three-dimensional spreading of the spray for each section may be determined. At 630, the dose placement may be optimized to maximize coverage and minimize time required. At 632 these steps may be repeated for all segments to be treated and a resulting aggregate treatment plan is determined. At 634, a user, such as a medical professional may review and approve of the treatment plan that was determined. At 636, the treatment plan may be output by software to the console.

At 638, the treatment may begin. At 640, the patient may be prepped for treatment. At 642 the robot or other navigation mechanism are set up. Intubation is performed as well. At 644, the user (e.g., operator of the system) inserts a catheter for delivering energy. The catheter may, for example, deliver a cryospray or other forms of ablation energy or mechanical energy. At 646, the user may select a target segment and confirms the selection. At 648, the console sends coordinates of the target for the robot. At 650, the catheter is maneuvered to the target location by the robot or other mechanism and alerts the console. At 652, the user confirms the location and delivers energy to the target via the catheter. At 654, the console logs that the dose has been delivered and queues the next target location (if any remain) for treatment. At 656, a check is made whether there are any more segments to be treated. If not, treatment is completed at 658. If so, a check is made at 660 if the oxygen level of the patient (e.g., saturated oxygen level) is above a threshold. If the oxygen level is high enough, the process repeats at 652 with a new segment. If the oxygen level is too low, at 662, the patient is oxygenated and then the process repeats at 652.

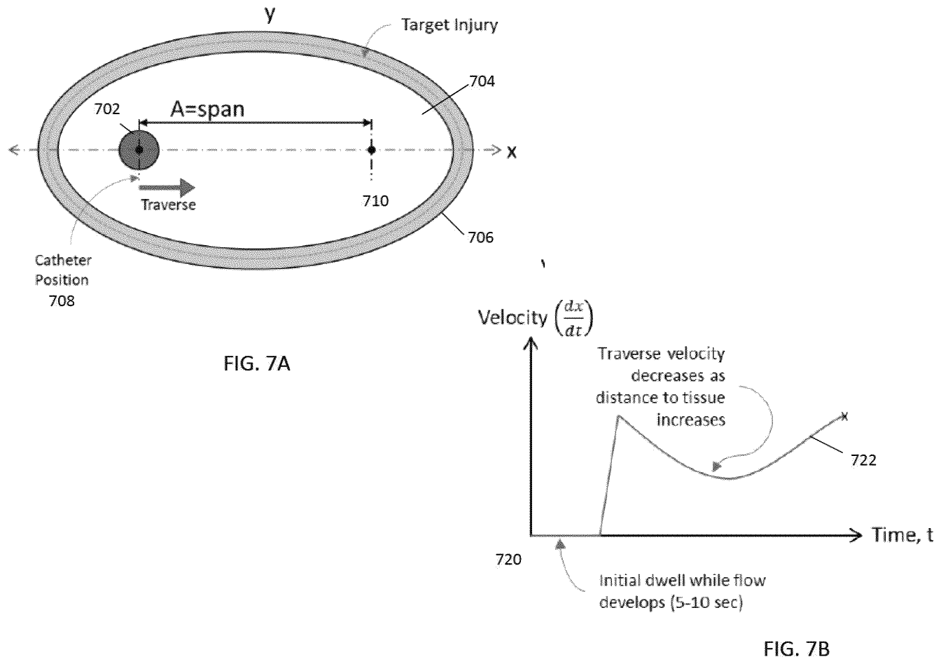

Instead of applying discrete doses (e.g., applying a dose at one location and then moving the catheter in a transverse manner across the lumen), the robotically-guided system may apply energy continuously while maneuvering to optimize energy uniformity throughout the cross section. For instance, FIG. 7A shows the catheter tip 702 positioned in the lumen 704 of a body part 706, like an airway. The catheter tip 702 is moved from a first centered location 708 to a second centered location 710. FIG. 7B shows a plot 720 of velocity over time (see curve 722). The plot 720 shows how the velocity of the catheter tip can be varied over time as the dose is applied—for example, to accommodate a ramp-up period and to slow down as the distance to the tissue increases (e.g., as the catheter moves closer to the geometric center of the body lumen).

It is also noted that that the catheter can be moved longitudinally forward and backward in the lumen while a dose is continuously applied. For example, some robotic catheter systems can be used to locate discrete points (e.g. for biopsies). However, existing systems have not been applied to execute a dynamic path or to reposition between energy applications in rapid succession, as described herein. Suitable systems, such as Intuitive Ion or Medtronic Superdimension may be used according to exemplary embodiments to execute 3-D CNC control of the catheter tip while applying energy. Software as described herein may accurately track the movement of the catheter relative to the patient in real-time as the energy is being applied.

A continuous pullback strategy can result in more complete goblet cell eradication. For instance, continuous pullback can cover full branch segments rather than discrete spaced rings, thus eliminating untreated margins between manually placed doses.

A continuous pullback strategy can also reduce each treatment session, in some cases by up to 14-15 minutes. Each spray loses approximately 5-10 seconds cooling the fluid path of the catheter before a spray can be applied (thus developing a predominately liquid stream), and 30 seconds between sprays as the system reacclimates and thaws the catheter. A continuous pullback strategy can reduce or eliminate these waiting times since the catheter is supplying cryogen for greater periods of time than in a discrete dose plan.

It is also noted that continuous longitudinal pullback can be combined with transverse movement to achieve complex treatment patterns (e.g., to avoid hazards).

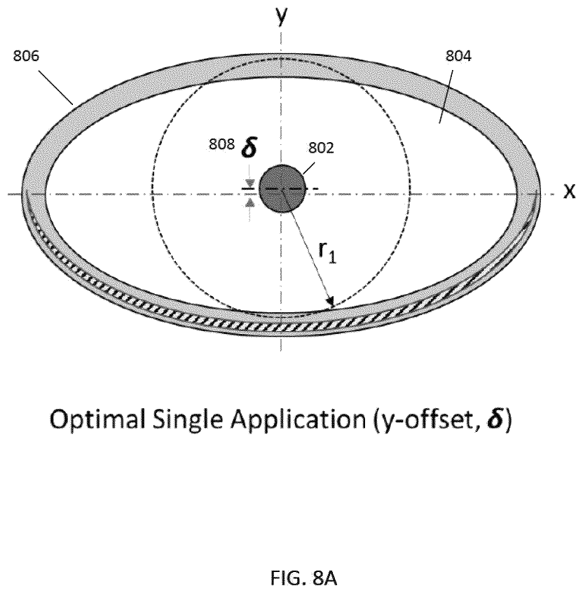

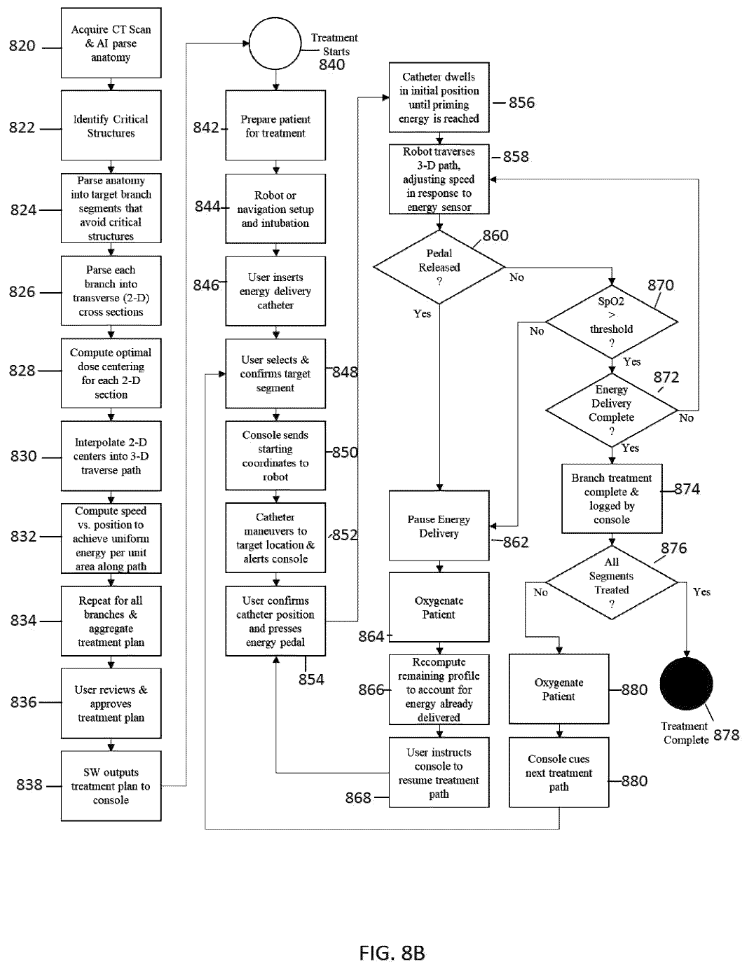

FIG. 8A depicts a catheter tip 802 arranged in the lumen 804 of a body part 806, like an airway, with a y-axis offset 808. FIG. 8B illustrates an example routine for applying a cryogenic fluid to a patient using a pullback strategy, as might be applied in the example shown in FIG. 8A. Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

At 820, a CT scan of the patient may be obtained, and AI may be used to parse out anatomical features. At 822, critical structure in the anatomy may be identified. At 824, the anatomy may be parsed into target branch segments that avoid critical structures. At 826, each branch may be parsed into transverse cross-sections. At 828, optimal dose centering may be computed for each 2-D section. At 830, the 2-D sections may be interpolated into a 3-D transverse path. At 832, speed versus position may be computed to achieve uniform energy per unit area along the path. At 834, this may be repeated for all of the branches and the results are aggregated. At 836, the user may review and approves the treatment plan. The treatment plan may be output to the console at 838.

At 840, treatment may start. At 842, the patient may be prepared for treatment. The robot or other navigation means may be set up, and intubation may be performed at 844. At 846, the user may insert the energy delivery catheter. At 848, the user may select a target segment and confirm the target segment. At 850, the console may send starting coordinates to the robot. At 852, the catheter may be maneuvered to the target location and may alert the console. At 854, the user may confirm the catheter position and may press an energy pedal. At 856, the catheter may dwell in its initial position until a priming energy level is reached. At 858, the robot may traverse a 3-D path. The speed may be adjusted in response to energy sensor. At 860, a check may be made whether the energy pedal is released. If so, at 862, energy delivery may be paused. At 864, the patient may be oxygenated. At 866, the remaining profile may be recomputed to account for the energy that has already been delivered, and at 868, the user may instruct the console to resume the treatment path. The process may then resume at 854.

If at 860, the pedal has not been released, at 870, a check is made of the oxygen level of the patient. If the oxygen level is below a threshold, the process shifts to 862. If the oxygen level is above a threshold, at 872, a check may be made of whether energy delivery is complete. At 874, if energy delivery is complete, then branch treatment is completed, and the completion may be logged by the console. At 876, a check may be made of whether all segments have been treated. If all segments have been treated, the treatment is complete at 878. If not, the patient may be oxygenated at 880, and the console may cue up the next treatment path at 880. The process may begin again at 848. If the energy delivery us not complete as checked at 872, the process continues again at 858.

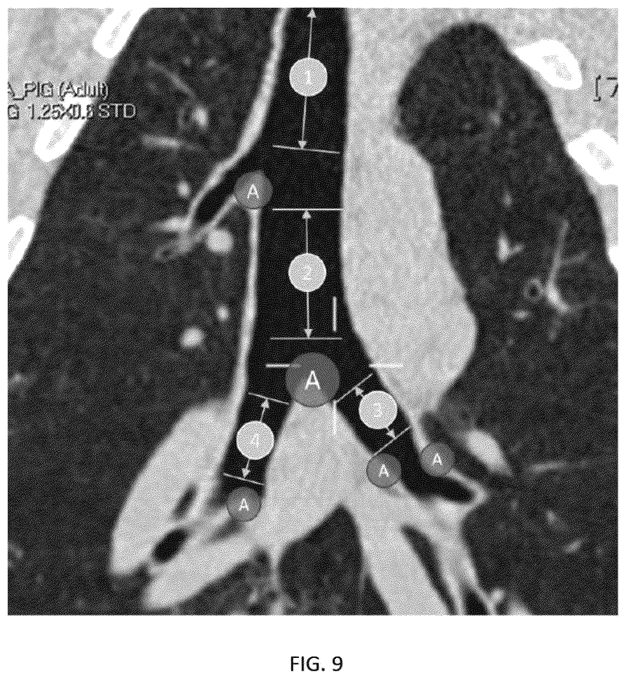

FIG. 9 depicts an example of a treatment plan that might be developed by the treatment planning software from a 3D structural map. In FIG. 9, areas marked “A” represent areas at which treatment should be avoided (e.g., due to structural risk). Paths 1, 2, 3, and 4 represent 2D linear paths which avoid structure, where each path can be treated with a continuous dose in a time of less than 90 seconds to allow reoxygenation between doses.

According to a simple treatment plan, it may be assumed that each segment (1-4) has a fixed diameter; thus, a 2-D traverse velocity may be held constant after an initial dwell period as described above.

However, because the system may make use of a 3D structural map, it may be possible to improve on this treatment plan to accommodate more complex geometries. For example, if it is known that each segment has a continuously varying diameter, then the planning software may alter the traverse velocity in a manner inversely proportional to the lumen caliber. The software may sense the underlying structure of each segment (from the 3D structural map) and define a three-dimensional traverse path through the segment to optimize coverage and avoid overtreatment. As previously noted, the path by which the catheter longitudinally traverses the lumen and the forward/backward motion of the catheter may be considered together in order to develop a more complex treatment plan. This may allow each section to be optimally treated, given the time (and other) constraints.

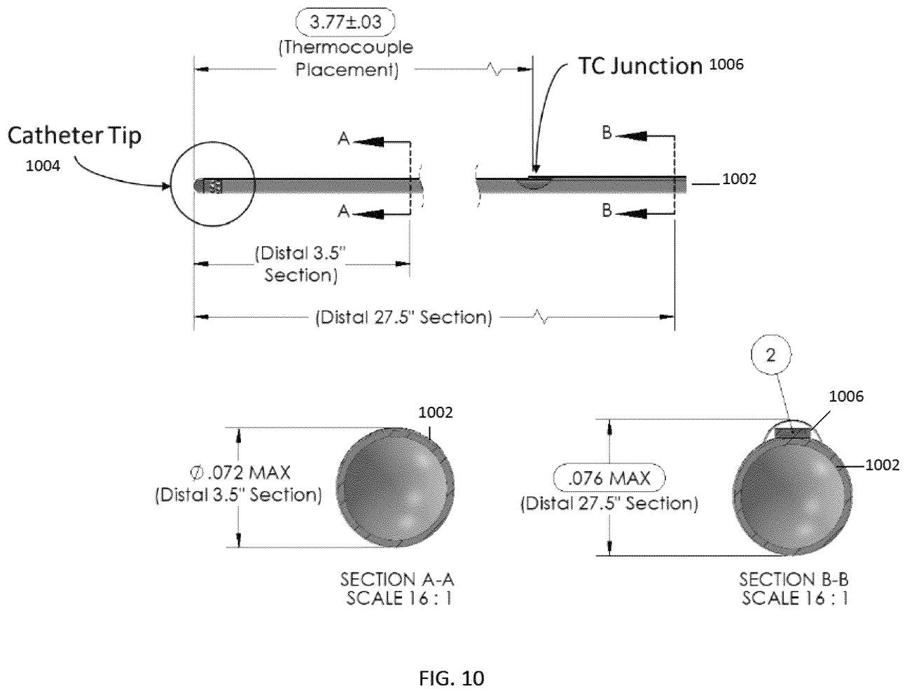

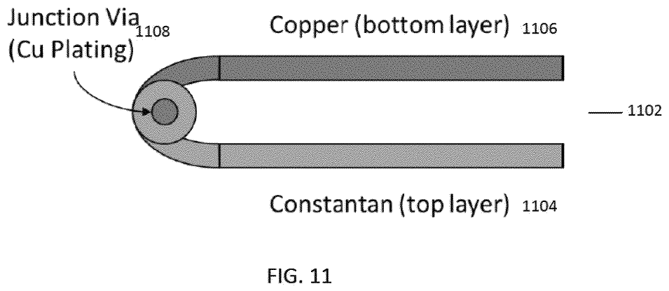

FIGS. 10-12 depict an example of an optional heater circuit for the catheter's energy sensor. FIG. 10 depicts the catheter 1002, with the catheter tip 1004 including a cryogenic spray nozzle on the left side. A thermocouple (FIG. 11) connects the catheter tip to a thermocouple junction 1006. Cross-sectional views of the catheter at Points A and B are also depicted.

The thermocouple 1102 shown in FIG. 11 is a relatively thin version that utilizes copper 1106 and constantan 1104 layers in a flexible circuit. A resistive constantan element is added radially around the thermocouple junction 1108, and two copper leads are provided parallel to the copper thermocouple leg.

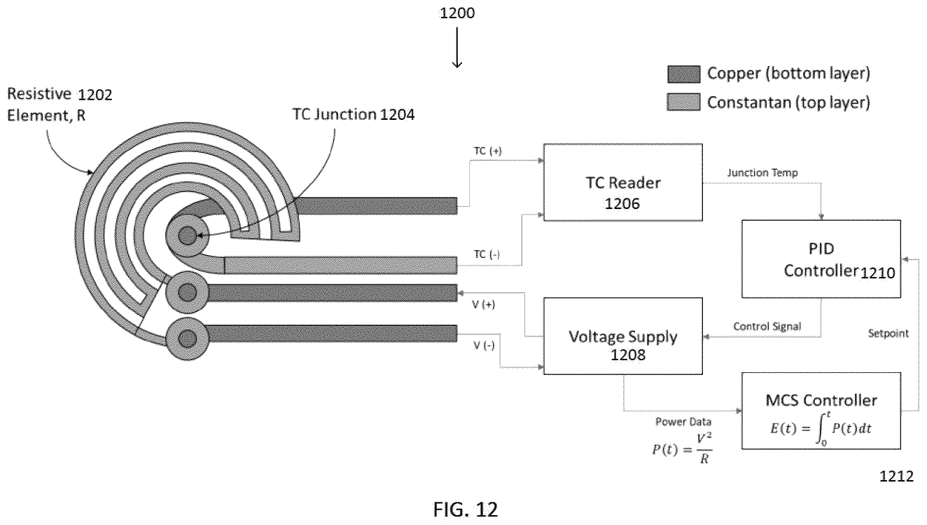

FIG. 12 depicts an example 2-layer isothermal energy sensor circuit 1200 including a resistor element 1202 (e.g. NiCr wire or Constantan) patterned around the temperature sensor junction 1204. The resistor circuit is electrically energized via two copper leads to avoid generating heat along the entire length of the catheter, concentrating the heating source radially inward toward the thermocouple junction. To reduce electrical cross-talk, an infrared fiber may optionally be applied to heat the junction (for example with a laser element located at the proximal end of the catheter). The circuit includes a TC reader 1206, a proportional integral derivative (PID) controller 1210, a voltage supply 1208 and an MCS controller 1212. As can be seen the MCS controller 1212 sets the setpoint for the PID controller 1210. The PID controller attempts to keep the junction temperature at the set point and send a control signal to the voltage supply 1208 to control the voltage applied to the resistive element 1202.

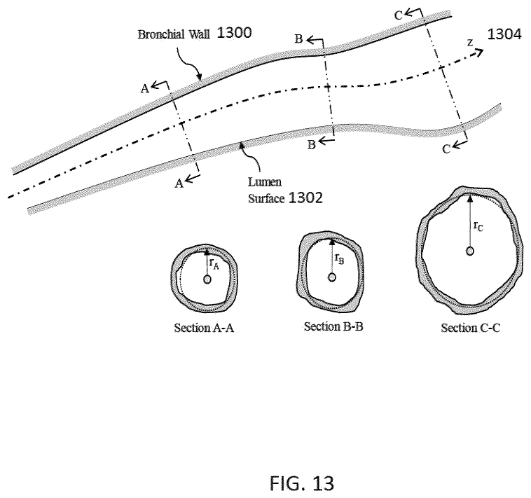

As was mentioned above, the software may determine an optimized axial traverse path for the catheter in treating the patient. FIG. 13 depicts an example of an optimized traversal path 1304 through the lumen of bronchi. The bronchial wall 1300 and the luminal surface 1302 are depicted. Cross-sections of section A-A, B-B, and C-C are shown and evidence that the lumen may be at vary radially along its length. The depicted branch is parsed into sections A-A, B-B, C-C, and the like. Each section may be processed to determine the ideal centering of the transverse path 1304 and to compute the target energy at each section along the path. While only three sections are depicted, many sections may be taken to create an effectively continuous path.

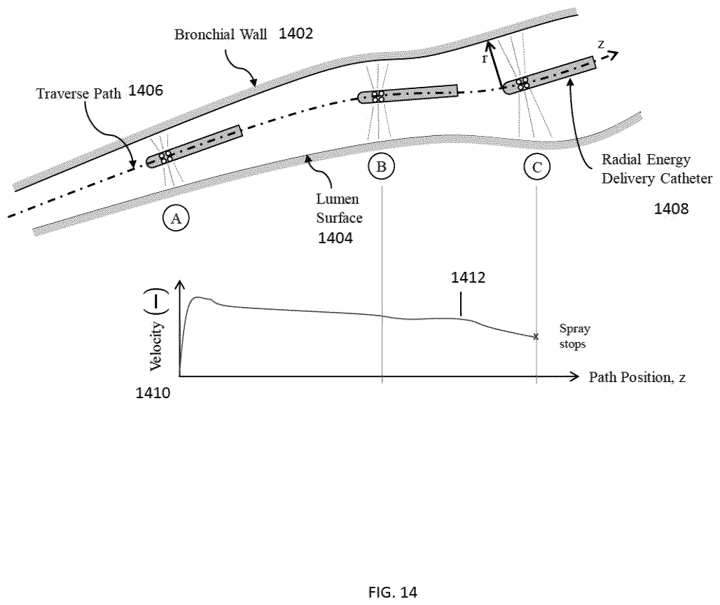

As shown in FIG. 14, the radial energy delivery catheter 1408, traverses along the optimized path 1406 through the lumen of the branch. Energy may be delivered to the lumen surface 1402 of the bronchial wall. The catheter delivers energy proportional to lumen size (i.e. radius) to maximize uniformity. As can be seen in curve 1412 shown in the companion plot 1410 of path velocity versus path position (note the designations for the cross-sections A, B, and C), the path velocity may be adjusted in real-time based on energy feedback. As also can be seen in the plot, the catheter dwells at point A.

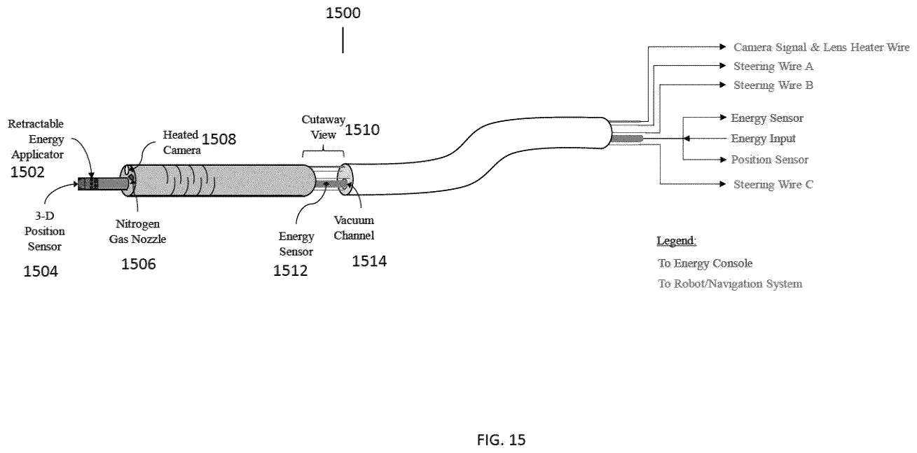

FIG. 15 depicts an example of the end of the catheter 1500 that is used to treat the patient. A retractable energy applicator 1502 may be provided to apply energy to the patient as described above. The energy applicator 1502 may be, for example, a radial spray device, an ultrasonic transducer, a light therapy component, a non-thermal pulsed field generator, or other form of energy output device. A 3-D sensor 1504 may be, for example, a position sensor that reports a 3-D path of the catheter or may be a shape-sensing fiber that reports the 3-D path. A heated camera 1508 may be provided to prevent lens fogging. A nitrogen gas nozzle 1506 may be provided to dry purge gas across and away from the lens to ensure that secretions do not block the receipt of images during treatment. A cutaway view shows an energy sensor 1512 and a vacuum channel 1514 that provides insulation. Steering Wires A, B, and C may be provided to control the navigation of the catheter via a joystick or other mechanism. Other wires are shown as well.

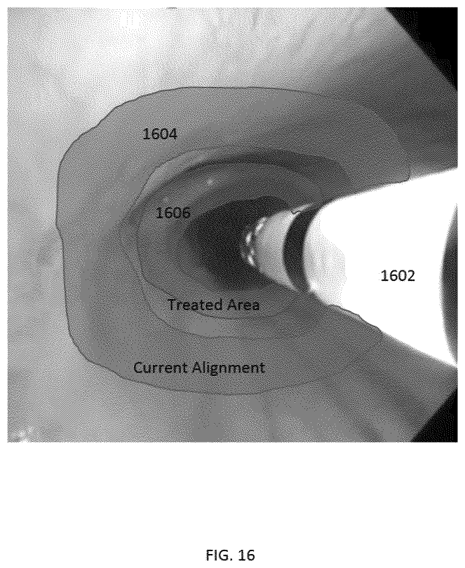

FIG. 16 depicts augmented reality features that may be provided in exemplary embodiments. When the tip of the catheter 1602 is repositioned, the system may display translucent overlays 1604 and 1606 to show the area that has been previously treated and a predicted treatment zone at the current location, respectively. The translucent overlays 1604 and 1606 are useful to the user in guiding the catheter for the next energy application. The overlays assist in both axial and radial positioning. The overlays 1604 and 1606 are generated by stitching together images based on 3-D coordinates to create a mesh map of the anatomy. The delivery of energy is recorded relative to the mesh map.

The purpose of this heater configuration is to variably apply heat to the temperature sensing junction in such a manner that the sensor is maintained at an approximately constant temperature throughout dose application. Without this heating element, the sensor would become “saturated” as it reaches the temperature of the energy source (e.g. cryogen) and would no longer respond to additional cooling energy application. By maintaining a constant temperature, the sensor can function continuously by measuring the energy input to the junction required to balance the cooling energy being applied to the junction.

The components and features of the devices described above may be implemented using any combination of discrete circuitry, application specific integrated circuits (ASICs), logic gates and/or single chip architectures. Further, the features of the devices may be implemented using microcontrollers, programmable logic arrays and/or microprocessors or any combination of the foregoing where suitably appropriate. It is noted that hardware, firmware and/or software elements may be collectively or individually referred to herein as “logic” or “circuit.”

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would be necessarily be divided, omitted, or included in embodiments.

At least one computer-readable storage medium may include instructions that, when executed, cause a system to perform any of the computer-implemented methods described herein.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

A procedure is here, and generally, conceived to be a self-consistent sequence of operations leading to a desired result. These operations are those requiring physical manipulations of physical quantities. Usually, though not necessarily, these quantities take the form of electrical, magnetic or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, the manipulations performed are often referred to in terms, such as adding or comparing, which are commonly associated with mental operations performed by a human operator. No such capability of a human operator is necessary, or desirable in most cases, in any of the operations described herein, which form part of one or more embodiments. Rather, the operations are machine operations. Useful machines for performing operations of various embodiments include general purpose digital computers or similar devices.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Various embodiments also relate to apparatus or systems for performing these operations. This apparatus may be specially constructed for the required purpose or it may comprise a general purpose computer as selectively activated or reconfigured by a computer program stored in the computer. The procedures presented herein are not inherently related to a particular computer or other apparatus. Various general purpose machines may be used with programs written in accordance with the teachings herein, or it may prove convenient to construct more specialized apparatus to perform the required method steps. The required structure for a variety of these machines will appear from the description given.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

Exemplary embodiments may include the following:

In one aspect, a method for treating a disorder of the bronchial tree includes generating an image of an area of the bronchial tree to be treated, generating a treatment plan of the area of the bronchial tree, the treatment plan includes a target location at which to apply a computed dose of treatment energy, steering a robotic catheter to the treatment location, the robotic catheter configured to deliver the treatment energy, and applying the computed dose of treatment energy at the target location.

The image may be a three-dimensional image obtained using at least one of a CT scan or MRI imaging.

The energy may include one or more of thermal energy, non-thermal ablation energy, a pulsed electrical field, irreversible electroporation, or dielectrophoresis.

Applying the computed dose of treatment energy may include continuously applying the treatment energy with the catheter while moving the catheter in a region including the target location.

The treatment location may include a wall of a body lumen, and the method may further include programmatically positioning the catheter within the body lumen based on the treatment plan.

The method may also include identifying a second target location as part of the treatment plan, and automatically maneuvering the robotic catheter to the second target location after applying the computed dose of treatment energy. Other technical features may be readily apparent to one skilled in the art from the following figures, descriptions, and claims.

In one aspect, a steerable catheter includes a video imager, a steerable tip, and a lens temperature controller.

The steerable tip may include at least one of a wire for actuating the steerable tip or a magnetic guidance system.

The steerable catheter may also include an infrared imager.

The steerable catheter may also include a thermocouple. The thermocouple may include an isothermal energy sensor including a resistive element.

In one aspect, a system includes a robotically steerable catheter. The system also includes a console that includes a processor, and a memory storing a treatment plan and instructions that, when executed, cause the processor to execute a treatment plan by maneuvering the robotically steerable catheter into positions defined by the treatment plan and applying one or more doses of energy at the positions.

The robotically steerable catheter may include an imager and a steering mechanism.

The instructions may be configured to receive image information from an imager that is not integral with the robotically steerable catheter.

The instructions may further be configured to steer the robotically steerable catheter via a robotic locomotion system separate from the catheter.

The system may also include a guided working channel through which the robotically steerable catheter is provided.

In one aspect, a method for treating a disorder of the bronchial tree includes generating an image of an area of the bronchial tree to be treated, displaying the image on a console display, overlaying the displayed image with a visual element indicating a previous dose applied to a treatment location, moving a catheter to the treatment location, the catheter configured to deliver treatment energy, and applying a dose of treatment energy at the target location.

The dose of treatment energy may be automatically applied based on a computed dosage amount determined by the console.

Moving the catheter to the treatment location may further include automatically moving the catheter using a robotic system based on a treatment plan stored at the console.

The method may also include receiving confirmation from a user of the console, and sending a command signal to apply the dose of treatment energy in response to receiving the confirmation, where the console is configured not to apply the dose in the absence of the command signal.

The method may also include generating a three-dimensional map of the bronchial tree while navigating the catheter using image recognition software. Other technical features may be readily apparent to one skilled in the art from the following figures, descriptions, and claims.

The three-dimensional map may be a mesh map.

The visual element may include a color filter based on one or more previous locations of the catheter during one or more previous dose applications.

The visual element may include one or more fiducial reference points based on one or more previous locations of the catheter during one or more previous dose applications.

The previous locations of the catheter may be determined by the console using one or more of an infrared history of the treatment location or a frost pattern at the treatment location that are indicative of locations where energy has been previously applied.

Other technical features may be readily apparent to one skilled in the art from the figures, descriptions, and claims.

What is claimed is:

1. A method for treating a disorder of the bronchial tree, comprising:
generating an image of an area of the bronchial tree to be treated;
generating a treatment plan of the area of the bronchial tree, the treatment plan comprising a target location at which to apply a computed dose of treatment energy;
steering a robotic catheter to the treatment location, the robotic catheter configured to deliver the treatment energy; and
applying the computed dose of treatment energy at the target location.

2. The method of claim 1, wherein the image is a three-dimensional image obtained using at least one of a CT scan or MRI imaging.

3. The method of claim 1, wherein the energy comprises one or more of thermal energy, non-thermal ablation energy, a pulsed electrical field, irreversible electroporation, dielectrophoresis, or mechanical energy.

4. The method of claim 1, wherein applying the computed dose of treatment energy comprises continuously applying the treatment energy with the catheter while moving the catheter in a region including the target location.

5. The method of claim 1, wherein the treatment location comprises a wall of a body lumen, and further comprising programmatically positioning the catheter within the body lumen based on the treatment plan.

6. The method of claim 1, further comprising identifying a second target location as part of the treatment plan, and automatically maneuvering the robotic catheter to the second target location after applying the computed dose of treatment energy.

7. A steerable catheter comprising:
a video imager,
a steerable tip, and
a lens temperature controller.

8. The steerable catheter of claim 7, wherein the steerable tip comprises at least one of a wire for actuating the steerable tip or a magnetic guidance system.

9. The steerable catheter of claim 7, further comprising an infrared imager.

10. The steerable catheter of claim 7, further comprising a thermocouple.

11. The steerable catheter of claim 7, wherein the thermocouple comprises an isothermal energy sensor including a resistive element.

12. A system comprising:
a robotically steerable catheter; and
a console comprising:
a processor; and
a memory storing a treatment plan and instructions that, when executed, cause the processor to execute a treatment plan by maneuvering the robotically steerable catheter into positions defined by the treatment plan and applying one or more doses of energy at the positions.

13. The system of claim 12, wherein the robotically steerable catheter comprises an imager and a steering mechanism.

14. The system of claim 12, wherein the instructions are configured to receiving image information from an imager that is not integral with the robotically steerable catheter.

15. The system of claim 12, wherein the instructions are configured to steer the robotically steerable catheter via a robotic locomotion system separate from the catheter.

16. The system of claim 12, further comprising a guided working channel through which the robotically steerable catheter is provided.

17. A method for treating a disorder of the bronchial tree, comprising:
generating an image of an area of the bronchial tree to be treated;
displaying the image on a console display;
overlaying the displayed image with a visual element indicating a previous dose applied to a treatment location;
moving a catheter to the treatment location, the catheter configured to deliver treatment energy; and
applying a dose of treatment energy at the target location.

18. The method of claim 17, wherein the dose of treatment energy is automatically applied based on a computed dosage amount determined by the console.

19. The method of claim 17, wherein moving the catheter to the treatment location comprises automatically moving the catheter using a robotic system based on a treatment plan stored at the console.

20. The method of claim 17, further comprising receiving confirmation from a user of the console, and sending a command signal to apply the dose of treatment energy in response to receiving the confirmation, wherein the console is configured not to apply the dose in the absence of the command signal.

21. The method of claim 17, further comprising generating a three-dimensional map of the bronchial tree while navigating the catheter using image recognition software.

22. The method of claim 21, wherein the three-dimensional map is a mesh map.

23. The method of claim 21, wherein the visual element comprises a color filter based on one or more previous locations of the catheter during one or more previous dose applications.

24. The method of claim 21, wherein the visual element comprises one or more fiducial reference points based on one or more previous locations of the catheter during one or more previous dose applications.

25. The method of claim 23, wherein the previous locations of the catheter are determined by the console using information that is indicative of locations where energy has been previously applied.

26. The method of claim 24, wherein the previous locations of the catheter are determined by the console using information that is indicative of locations where energy has been previously applied.
